(19) United States
(12) Patent Application Publication
(10) Pub. No.: US 2023/0363929 A1
(43) Pub. Date: Nov. 16, 2023

(54) Implantable Joint Prosthesis with Integrated Biasing Systems

(71) Applicant: Extensor, LLC, HOUSTON, TX (US)

(72) Inventors: GREGORY W STOCKS, HOUSTON, TX (US); MARK R BRINKER, HOUSTON, TX (US)

(73) Assignee: Extensor, LLC, HOUSTON, TX (US)

(21) Appl. No.: 18/184,638

(22) Filed: Mar. 15, 2023

Related U.S. Application Data
(63) Continuation-in-part of application No. 17/187,795, filed on Feb. 27, 2021.
(60) Provisional application No. 63/269,401, filed on Mar. 15, 2022.
(60) Provisional application No. 63/049,612, filed on Jul. 8, 2020.
(60) Provisional application No. 62/983,560, filed on Feb. 28, 2020.

(51) Int. Cl. A61F 2/48

(52) U.S. Cl. CPC A61F 2/484; A61F 2002/30079

(57) ABSTRACT

An implantable joint prosthesis simulating an elbow joint, an ankle joint, a shoulder joint or a hip joint, the implantable prosthesis having one or more integrated biasing system(s) configured to bias the prosthetic joint to one or more preferred orientations or alignments.

### CROSS REFERENCE TO RELATED APPLICATIONS

The disclosure in this application is based on and wholly incorporates by reference for all purposes the disclosure in commonly owned U.S. Pat. Application Serial No. 17/187,795 entitled Implantable Prosthetic Knee Joint with Integrated Extensor Mechanism, filed 2021-02-27, which claims benefit of and priority to U.S. Provisional Pat. Application serial number 63/269,401 entitled Implantable Joint Prosthesis with Integrated Biasing Systems, filed 2022-03-15.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

Field of the Invention. The inventions disclosed and described herein relate to an implantable joint prosthesis for patients with compromised or non-existing joint muscle systems, such as extensor, abductor, adductor, or other joint muscle systems.

### Description of the Related Art

In addition to the art described in U.S. Pat. Application Serial No. 17/187,795, which is incorporated herein, joints other than knee joints with compromised muscle systems also made need replacement with other than external prostheses. For example and not limitation, elbow joints, ankle joints, shoulder joints, and hip joints will benefit from the inventions disclosed and described herein.

The present inventions are directed to improvements in implantable total joint prostheses for use with patients with compromised or non-existent joint musculature systems.

### BRIEF DESCRIPTION OF THE SEVERAL VIEWS OF THE DRAWINGS

The following figures form part of the present specification and are included to demonstrate further certain aspects of the present invention. Our inventions may be better understood by reference to one or more of these figures in combination with the detailed description of specific embodiments presented herein. However, neither the detailed description nor the figures are intended or provided to limit the scope of the claims.

FIG. 1 illustrates a typical human knee.

FIG. 2 illustrates the extensor mechanism of a typical human knee.

FIG. 3 illustrates an in situ total knee prosthesis having an integrated prosthetic extensor mechanism for persons with compromised extensor mechanisms.

FIG. 4 illustrates an in situ intermedullary knee prosthesis having an integral extensor mechanism for persons with compromised extensor systems.

FIG. 5 illustrates another embodiment of an in situ total knee prosthesis having an integrated extensor mechanism for persons with compromised extensor systems.

FIG. 6A illustrates an embodiment of a smart prosthetic knee and associated technology.

FIG. 6B illustrates an embodiment of a control system for smart prosthesis.

FIG. 7 illustrates a logic flow chart for a top-level algorithm for software useful with embodiments of the present inventions.

FIG. 8 illustrates an embodiment of a rotation mechanism for an in situ total knee prosthesis having an integrated extensor mechanism for persons with compromised knee extensor systems.

FIG. 9 illustrates another embodiment of an in situ total knee prosthesis having an integrated extensor mechanism for persons with compromised extensor systems.

FIG. 10 illustrates an elbow prosthesis utilizing aspects of the disclosed inventions.

FIG. 11 illustrates possible embodiments of an implantable elbow joint prothesis utilizing aspects of the disclosed inventions.

FIG. 12 illustrates an implanted ankle prosthesis.

FIG. 13 illustrates an ankle prosthesis utilizing aspects of the disclosed inventions.

FIG. 14A illustrates a shoulder prosthesis utilizing aspects of the disclosed inventions.

FIG. 14B illustrates a reverse shoulder prosthesis utilizing aspects of the disclosed inventions.

FIG. 15 illustrates a hip prosthesis utilizing aspects of the disclosed inventions.

FIG. 16 illustrates multiple embodiments of smart prosthetic joints and associated technology.

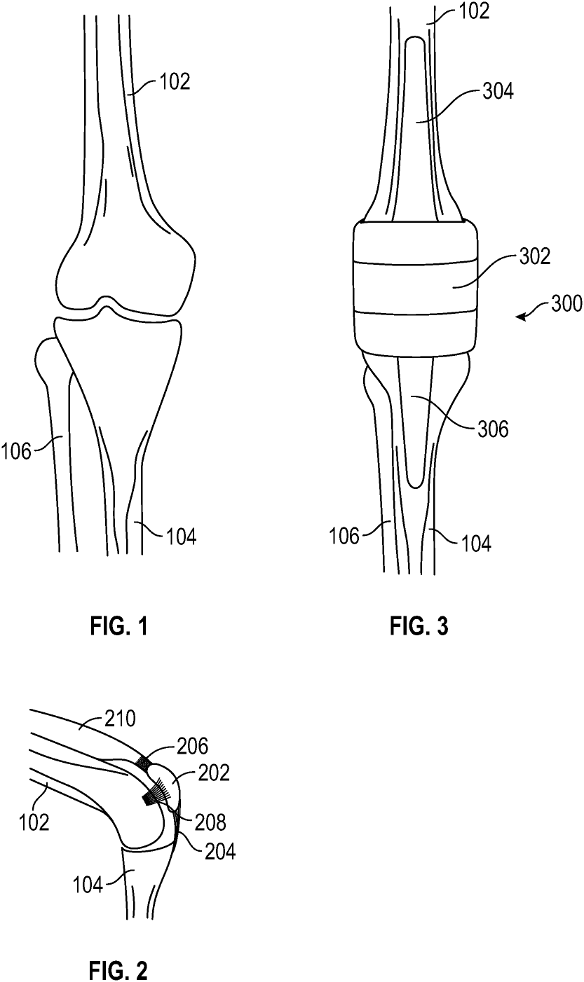

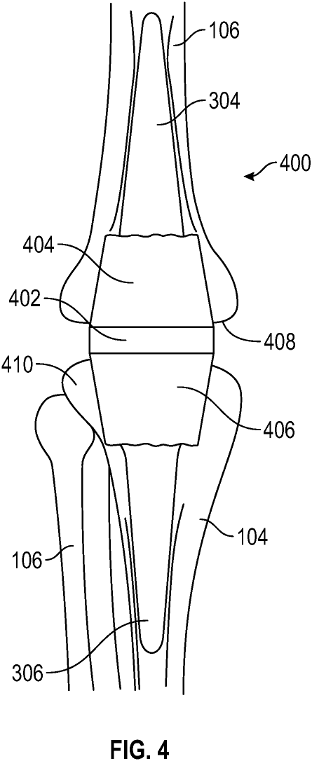

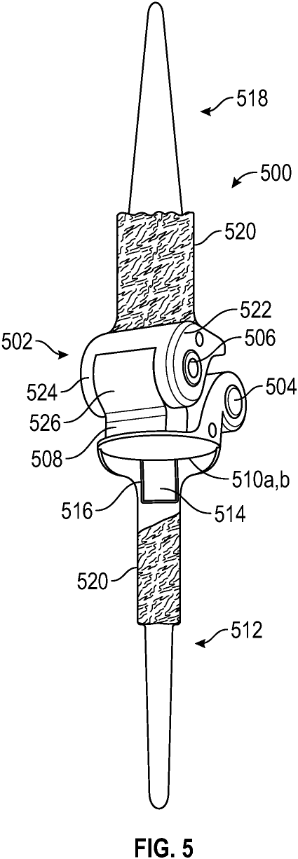

While the inventions disclosed herein are susceptible to various modifications and alternative forms, only a few specific embodiments have been shown by way of example in the drawings and are described in detail below. The figures and detailed descriptions of these specific embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and use the inventive concepts.

### DETAILED DESCRIPTION

The Figures described above, and the written description of specific structures and functions below are not presented to limit the scope of what we have invented or the scope of the issued claims. Rather, the Figures and written description are provided to instruct any person skilled in the art to make and use the inventions for which patent protection is sought. Those persons skilled in the art will appreciate that not all features of a commercial embodiment of the inventions are described or shown for the sake of clarity and understanding. Persons of skill in this art will also appreciate that the development of an actual commercial embodiment incorporating aspects of the present inventions will require numerous implementation-specific decisions to achieve the developer’s ultimate goal for the commercial embodiment. Such implementation-specific decisions may include, and likely are not limited to, compliance with system-related, business-related, government-related, and other constraints, which may vary by specific implementation, location and from time to time. While a developer’s efforts might be complex and time-consuming in an absolute sense, such efforts would be, nevertheless, a routine undertaking for those of skill in this art having benefit of this disclosure. It must be understood that the inventions disclosed and taught herein are susceptible to numerous and various modifications and alternative forms. Lastly, the use of a singular term, such as, but not limited to, “a,” is not intended as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the Figures and are not intended to limit the scope of the invention or the appended claims.

Aspects of the inventions disclosed herein may be embodied as an apparatus, system, method, or computer program product. Accordingly, specific embodiments may take the form of an entirely hardware embodiment, an entirely software embodiment or an embodiment combining software and hardware aspects, such as a “circuit,” “module” or “system.” Furthermore, embodiments of the present inventions may take the form of a computer program product embodied in one or more computer readable storage media having computer readable program code.

Items, components, functions, or structures in this disclosure may be described or labeled as a “module” or “modules.” For example, but not limitation, a module may be configured as a hardware circuit comprising custom VLSI circuits or gate arrays, off-the-shelf semiconductors such as logic chips, transistors, or other discrete components. A module also may be implemented as programmable hardware devices such as field programmable gate arrays, programmable array logic, programmable logic devices, or the like. Modules also may be configured as software for execution by several types of processors. A module of executable code may comprise one or more physical or logical blocks of computer instructions that may be organized as an object, procedure, or function. The executables of a module need not be physically located together but may comprise disparate instructions stored in various locations that when joined logically together, comprise the module and achieve the stated purpose or function. A module of executable code may be a single instruction, or many instructions, and may even be distributed over several different code segments, among different programs, and across several memory devices. Similarly, data may be identified and illustrated herein within modules, and may be embodied in any suitable form and organized within any suitable type of data structure. The data may be collected as a single dataset or may be distributed over various locations including over different storage devices, and may exist, at least partially, merely as electronic signals on a system or network. Where a module or portions of a module are implemented in software, the software portions may be stored on one or more computer readable storage media.

Reference throughout this disclosure to “one embodiment,” “an embodiment,” or similar language means that a particular feature, structure, or characteristic described in connection with the embodiment is included in at least one of the many possible embodiments of the present inventions. The terms “including,” “comprising,” “having,” and variations thereof mean “including but not limited to” unless expressly specified otherwise. An enumerated listing of items does not imply that any or all the items are mutually exclusive and/or mutually inclusive, unless expressly specified otherwise. The terms “a,” “an,” and “the” also refer to “one or more” unless expressly specified otherwise.

Furthermore, a described feature, structure, or characteristic of one embodiment may be combined in any suitable manner to create one or more other embodiments. In the following description, numerous specific details are provided, such as examples of programming, software modules, user selections, network transactions, database queries, database structures, hardware modules, hardware circuits, hardware chips, etc., to provide a thorough understanding of embodiments of the disclosure. Those of skill in the art having the benefit of this disclosure will understand that the inventions may be practiced without one or more of the specific details, or with other methods, components, materials, and so forth. In other instances, well-known structures, materials, or operations are not shown or described in detail to avoid obscuring aspects of the disclosure.

The description of elements in each Figure may refer to elements of proceeding Figures. Like numbers refer to like elements in all figures, including alternate embodiments of like elements. In some possible embodiments, the functions/actions/structures noted in the figures may occur out of the order noted in the block diagrams and/or operational illustrations. For example, two operations shown as occurring in succession, in fact, may be executed substantially concurrently or the operations may be executed in the reverse order, depending upon the functionality/acts/structure involved.

In general, our inventions relate to implantable joint prostheses for individuals that have compromised or non-functional joint muscle systems. For example, and not for limitation, the prostheses of the present inventions may comprise a modified conventional ex situ or external prosthetic knee joint with or without an integrated extensor mechanism configured to provide one or more biasing forces to the joint to simulate or approximate a level of normal knee function (e.g., extension). Additionally, and without limitation, the prostheses of the present inventions may comprise a modified conventional or unconventional implantable elbow joint prosthesis, ankle joint prothesis, shoulder prosthesis or hip joint prosthesis, with one or more integrated biasing systems structured to move or bias the joint to one or more preferred alignments or orientations.

The prostheses of the present inventions also may comprise an integrated braking mechanism (i.e., a system to retard, minimize or eliminate velocity and/or acceleration or both) configured to provide one or more constant or variable forces to the joint to simulate or approximate a level of human joint function.

Suitable biasing and/or braking mechanisms include, without limitation, spiral springs, wafer springs, hydraulic cylinders, pneumatic cylinders, magnets, camming surfaces, and linear motors. Implantable joint biasing systems or components may comprise torsion springs integrated into one or more joint hinges. Alternately, biasing systems components may comprise hydraulic, pneumatic, magnetic, or mechanical systems. In yet another envisioned embodiment, combinations of these extension biasing components may be combined. As described, the neutral spring condition with increasing amount of return biasing force from increasing amounts of flexion or movement may be combined with, as a non-limiting example, a magnetic component that negates some of the return biasing force to provide a limited or dampened return swing.

The biasing components may comprise a brake that releasably “locks” the implantable joint in particular position or range of positions yet is overcome by the swing forces associated with a normal or active movement.

A preferred biasing system comprises an assembly of magnets or magnets and magnetic materials. These biasing systems may comprising permanent magnets that attract each other, permanent magnets, such as ferromagnets, that repel each other, a combination of permanent magnets and paramagnetic material that attract one another; a combination of permanent magnets and diamagnetic material that repel one another; and combinations of permanent magnets, paramagnets, diamagnets, antiferromagnets and/or ferrimagnets alone or in combination with out biasing or braking systems to create a desired prosthetic joint operation. It will be appreciated that these systems can be configured to produce rotational attraction or repulsion as well as planar attraction or repulsion.

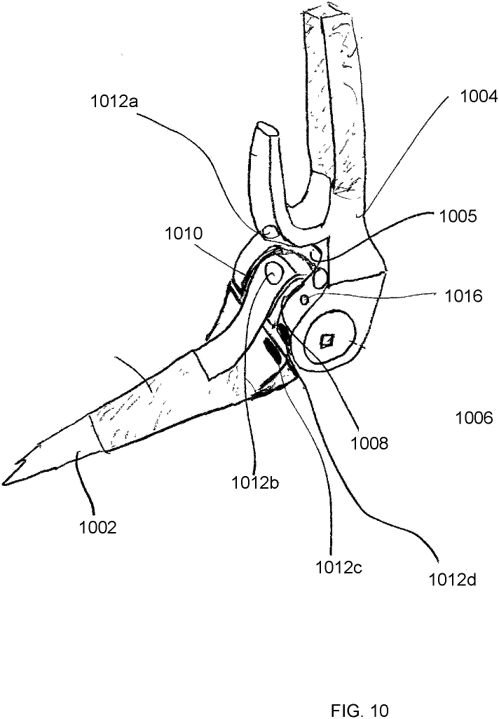

Thus, based on the disclosure and teaching found in U.S. Pat. Application Serial No. 17/187,795 and the disclosure and the teaching provided in this application, FIG. 10 illustrates a conventional, implantable elbow joint prothesis 1000 comprising an ulnar component 1002, a humeral component 1004, and a single axis rotation joint 1006 therebetween. The prosthesis 1000 also comprises a humeral bearing pad, and two ulnar bearings 1008, 1010. Not found in conventional elbow joint protheses, the prosthesis of FIG. 10 comprises an integrated biasing system structurally configured to move, urge, or bias the relative alignment of the humeral 1004 and ulnar 1002 components to a desired condition or state.

One type of biasing system suitable for use with the prothesis illustrated in FIG. 10 comprises one or more magnets 1012a-1012d (e.g., permanent magnets) placed in the prosthesis components to attract the components to a desired condition or state, or to repel the components to or from a desired condition or state. For example, if for a given patient it is preferred that the elbow joint be biased to a particular angular position, such as 45, 60 or 90 degrees flexion, magnets may be implanted in the components 1002, 1004 in such positions that the components are attracted to this preferred alignment or repelled to this preferred alignment. It will be appreciated that multiple magnetic biasing systems can be implemented, such as, for example, to bias the joint to full extension (0 degrees) and to 90 degrees by using two sets of magnets integrated into the prosthesis.

Because most implantable prosthesis are fabricated from non-magnetic (on a macro scale) alloy systems, it is contemplated that a magnetic biasing system may comprise one or more rods, buttons, pads, or other geometrically shaped permanent magnets that can be integrated into the non-magnetic alloy. For example, for rod shaped magnets, a hole corresponding to the size (e.g., diameter and length) of the magnetic rod can be drilled into the prosthetic component in the appropriate location or locations. The magnetic rod can be placed, preferably by an interference fit, into the hole. If necessary, such for biocompatibility issues, the magnetic rod can be covered in the hole, such as by an interference fit cap or cover, threaded cap, or welded cap of biocompatible base material. Persons of skill will now appreciate that any geometrically shaped magnet may be integrated into a prosthetic joint component as described, including casting the magnet systems into the joint components.

Permanent magnet systems suitable for use with the prostheses disclosed herein may compromise Ferrite magnets, Alnico magnets, Permalloy magnets, Neodymium magnets, and Samarium Cobalt magnets. The magnetic direction of the magnet(s) integrated into the prosthetic components can be configured to provide the desired amount and/or range of bias for the joint. For example, for an elbow prosthesis, aligning the magnetic directions between magnet(s) in the ulnar component and magnet(s) in the humeral components may provide a large, focused attractive force. In contrast, orienting the magnetic directions of the magnet systems may provide a more diffuse or unfocussed repelling force.

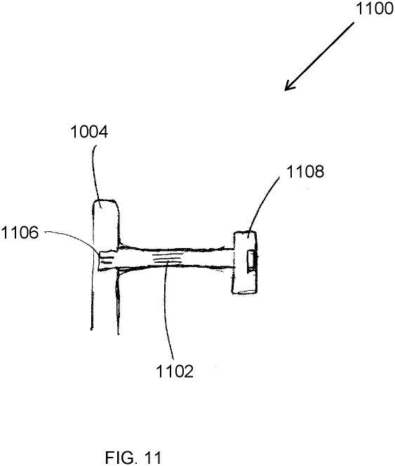

An alternative biasing system is illustrated in FIG. 11 (and FIG. 10). For a simple hinge joint prosthesis, like an elbow prosthesis, a biasing system may comprise a torsion spring 1100 that also functions as the axis of rotation. In the embodiment illustrated in FIG. 11, a torsion spring 1102 may comprise a rod anchored or attached at one end of the prosthesis, such as, for example, one side of the humeral component 1004. A mid portion of the torsion rod may have a splined or shaped cross section 1104 configured to structurally engage with the ulnar component 1002. Before or during implantation, the joint can be assembled such that the null condition of the torsion spring is the desired null position of the joint, for example, 60 degrees of flexion. As the joint increases or decreases flexion from the null position, the torsion spring will bias the joint back to the null position.

In one embodiment of a torsion spring suitable for use with implantable prostheses, a spring may comprise a splined or shaped end 1106 for non-rotating engagement with a portion of the humeral component. The other end of the spring may comprise a hub, such a disk-shaped portion to which the spring is affixed or integrated. As shown in FIG. 10, another portion of the humeral component has an opening 1014 configured to receive the spring hub 1108. The joint can be assembled by inserting the spring through the opening in the humeral opening, through the ulnar bearing components, the ulnar component and into the receptable in the humeral component as shown in FIG. 11. A lock screw 1016, as illustrated in FIG. 10 can secure the spring hub to the humeral component. Alternately, the spring force can be adjusted by no securing the hub to the femoral component and relying only on the shaped end of the torsion spring to react the biasing force.

Still further, depending on the amount and/or type of bias desired for a specific implantation, one or more positional (e.g., flexion) detents, friction surfaces or camming surface can be employed to favor or disfavor particular orientations, such as degrees of flexion or rotation.

It will be appreciated that the biasing system illustrated in FIG. 11 may be combined with other biasing or braking systems, such as, but not limited to, a magnet system that further attracts or repels the joint from the null or biased condition.

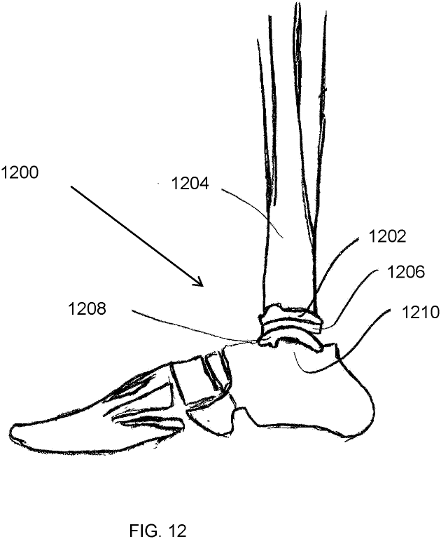

Expanding upon the disclosure presented above in the context of an elbow joint prosthesis, our inventions can benefit ankle joint prostheses as well. Illustrated in FIG. 12 is a conventional implantable ankle prothesis 1200 comprising a tibial component 1202 implanted in the tibia 1204, including a pad component 1206, and a talar component 1208 implanted in the talus 1210.

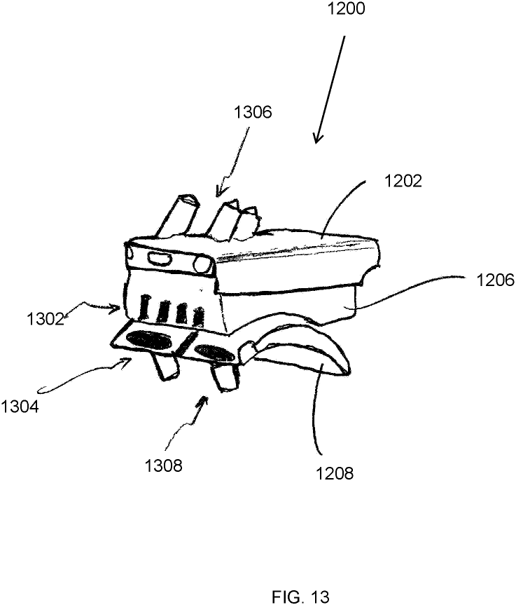

FIG. 13 illustrates an ankle prosthesis 1200 utilizing aspects of the inventions disclosed herein. For illustration, it may be desired to bias a compromised ankle joint to a flexion angle of, for example 90 degrees, one or more magnets 1302 may be embedded or formed the pad component 1206 and corresponding magnets 1304, or material susceptible to magnetic fields may be embedded or integrated in the talar component 1208 to bias the ankle joint to the desired flexion condition. Alternately, the magnets can be embedded, as described above, in modified portions of the tibial component and/or modified talar component. Also as disclosed above, multiple magnet systems or other biasing or braking systems can be utilized to provide one or more bias point of attraction or repulsion (planar or rotational).

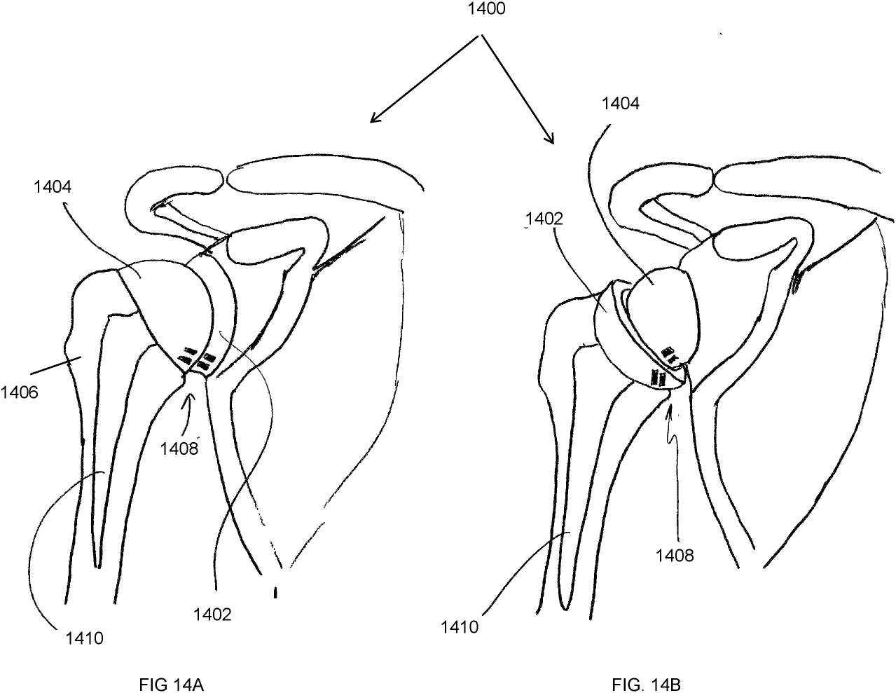

FIGS. 14A and 14B illustrate embodiments of implantable shoulder prothesis 1400 utilizing aspects of the inventions disclosed herein with respect to knee prostheses, elbow prostheses, and ankle protheses. Regardless of whether the shoulder prothesis 1400 is implanted in a conventional orientation in which the cup 1402 is located in the glenoidal cavity, and the associated ball 1404 is located on the end of the humerus 1406, FIG. 14A, or a reverse orientation as illustrated in FIG. 14B, one or more of the biasing or braking systems disclosed herein may be implemented.

For example, if it is desired to bias adduction of the shoulder joint, a magnetic biasing system 1408 may be embedded in the appropriate components. As illustrated in FIGS. 14A and 14B, rod-shaped magnets are embedded, as described previously, in both the ball and the cup to cause through magnetic attraction adduction of the arm. Alternately, magnets can be embedded, for example, on opposite sides of the ball and cup to repel the arm in adduction. In some embodiments, it may be desirable to provide a ring, or a portion of the ring on an outer portion of the cup to house the magnets associated with the up. For example, a non-load bearing ring of plastic, such as HDPE or UHWMPE may surround the periphery of the cup into which magnets are embedded or housed at the appropriate location or locations to achieve the desired bias of the joint.

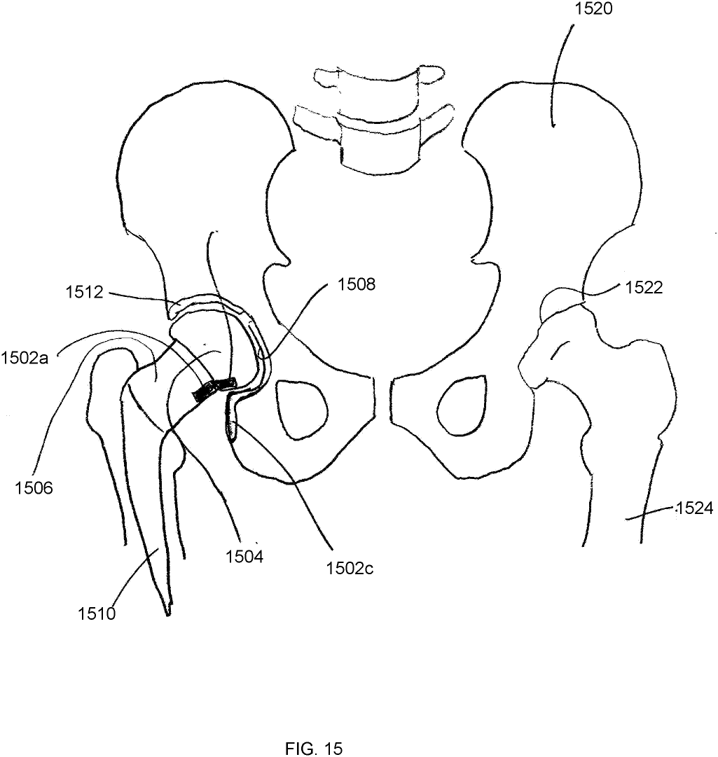

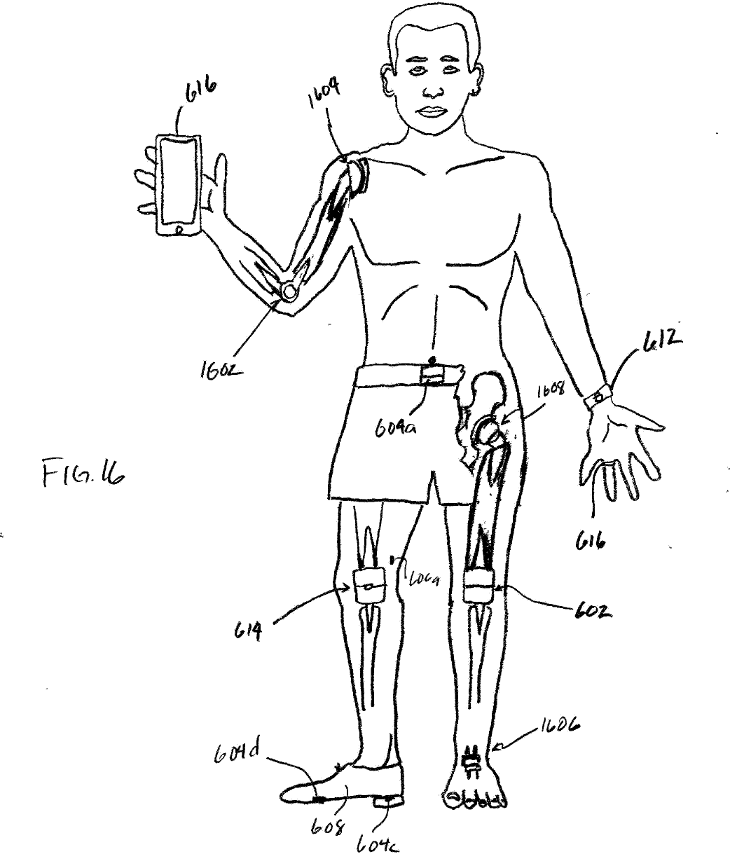

FIG. 15 illustrates an implantable hip prosthesis 1500 having a biasing system as disclosed and described herein. FIG. 15 shows one embodiment in which one or more magnets 1502 are embedded in the femoral ball 1504 or shank portion 1506 and one or more magnets 1502b embedded in the cup 1508, and/or non-load bearing ring about the cup or portion of the prothesis designed and configured to house the biasing magnets. While the implantable prosthesis 1500 illustrated in FIG. 15 is the conventional orientation, those of skill will understand that reverse orientation hip prosthesis can utilize the biasing systems disclosed herein.

In all of the embodiments disclosed herein that utilize magnetic systems as discussed above, it is preferred that the magnetic material, whether permanent, diamagnetic, paramagnetic, ferromagnetic, ferrimagnetic, or antiferromagnetic, or a combination, be structurally embedded in the implantable substrate, such as a titanium alloy or stainless steel, and be sealed, such as by welding or brazing therein against biomedical infiltration and compromise.

Although we have described specific embodiment of elbow, ankle, shoulder and hip joints with magnet-based biasing or braking systems, those of skill will appreciate that any one or more of the biasing or braking systems or mechanisms described for any of the prosthetic joint herein (i.e., knee, elbow, ankle, shoulder and/or hip) can be implemented in any other prosthetic joint. For example and not limitation, the hydraulic biasing system described with respect to the knee prosthesis herein, may be adapted and utilized on an ankle prosthesis, an elbow prosthesis, a shoulder prosthesis, or a hip prosthesis without departing from the scope of the inventions we have created.

We envision additional embodiments of the implantable prosthetic joints disclosed herein that make use of the features of a “smart” or microprocessor-based prosthetic system. In one of many possible embodiments that may be envisioned by those having benefit of this disclosure, a “smart” joint may be configured to communicate with wearable sensors, such as accelerometers, position sensors, angle sensors, pressure sensors, and/or load sensors to self-adjust ranges, limits and/or functions of the implanted prosthetic joint (or joints) for a particular person and/or for particular situations.

In several non-limiting embodiment illustrated in FIG. 16, a “smart” implantable prosthetic joint system 602 (knee), 1602 (elbow), 1604 (shoulder), 1606 (ankle) and/or 1608 (hip) may receive inputs from wearable sensors, such as one or more sensors in a person’s belt 604a, clothing 604b, shoes 604c, 604d or the like to set ranges and limitations of the system 602. These articles of clothing have the advantage that they are normally worn at specific locations relative to the prothesis and for specific activities. Placing sensors in belts, shoes and/or clothing is also much less encumbering than having to wear a sensor that would have to be applied at a specific location on a body. However, the inventions disclosed herein may make use of sensors removably or permanently attached to the body. For example and not limitation, implanted sensors with muscles that indicate muscle activation and direction also may be used with the prostheses contemplated herein.

In this envisioned embodiment, sensors in the belt 604a may relay pressure changes such as may be a contraction of a dorsi or gluteal muscle that may be a precursor to a push off step if the shoe sensor identifies that the legs are fully extended - e.g., the ipsilateral shoe is at a maximum distance below, e.g., the “smart” knee 602 and the contralateral shoe is appropriately nearby as would be normal for a stance. A small pressure change detected along the belt may indicate that the wearer is preparing to take a small or normal step. However, a larger pressure change detected may indicate that the wearer is preparing for a leap or lunge. The “smart” prosthesis 602 (knee), 1602 (elbow), 1604 (shoulder), 1606 (ankle) and/or 1608 (hip), may then process these signals and make instantaneous adjustments to its limits and ranges in anticipation of this activity.

In another embodiment, the collection of sensors may indicate that the shoes 604c, 604d are nearing the belt 604a. If the belt 604a is substantially vertically above the shoes, the “smart” knee 602 may anticipate that the wearer is crouching or perhaps preparing to sit. However, if the belt is offset from the shoes, it may be anticipated that the wearer is preparing to kneel. Algorithms may be implemented in the smart prothesis 602 (knee), 1602 (elbow), 1604 (shoulder), 1606 (ankle) and/or 1608 (hip) to temporarily adjust an amount of friction, resistance, or bias to the anticipated movement. Appropriate adjustments may be made to the ranges, limits, and even to the biasing of the “smart” joint for these interpreted sensor inputs.

In yet another envisioned embodiment, the pressures of the fronts and backs of the soles of the feet 604c, 604d in relationship to the distance between the feet may indicate that the wearer is walking or running. Greater pressure differences and longer strides may indicate running, which may require a larger extension bias than a normal walking stride. Similarly, sensing that one shoe has no sole pressure 604d while also sensing a rise in elevation may indicate the need to make adjustments in one or more of the smarts joint 602 (knee), 1602 (elbow), 1604 (shoulder), 1606 (ankle) and/or 1608 (hip).

While the examples given here are for sensors in a belt and shoes in relation to the prosthetic knee, Applicants envision embodiments with other sensors worn or placed on the body and used in relation to other implantable smart joints 602 (knee), 1602 (elbow), 1604 (shoulder), 1606 (ankle) and/or 1608 (hip). Without limitation, these may include appropriate sensors in wrist watches 612, rings 616, bracelets 612, necklaces, piercings, and any other device that may be worn on the body. Sensors that rely on a distance from a “smart” joint should maintain a relatively stable position relative to the body or relative to the joint or joints they support. Embodiments of inventions described herein may include sensors that monitor and relay other information such as, but not limited to, temperature, direction of gaze, respiration, heartbeat (all of which may be used as indicators of upcoming movements requiring smart joint adjustment).

Smart joints, such as 602 (knee), 1602 (elbow), 1604 (shoulder), 1606 (ankle) and/or 1608 (hip), may communicate with nearby sensors through means known to those of ordinary skill in the art. These communications pathways may include, without limit, near field communication (NFC) networks, Bluetooth including Bluetooth low energy (BLE), and several other radio frequency pathways.

Applicants envision that some people may have multiple “smart” joints 602, 614 (knees), 1602 (elbows), 1604 (shoulders), 1606 (ankles) and/or 1608 (hips) and that it may be beneficial for the “smart” joints to communicate with each other. As an example, but without limitation, a first “smart” knee 602 may work in tandem with a second smart knee 614. Or a first smart shoulder 1604 may work in tandem with a second smart shoulder (not shown) One or more sensors embedded in the first and second smart prosthetic may allow each smart joint to monitor the activity (including inactivity) of the other joint to for use by the movement algorithm in creating the desired amount of functionality (e.g., resistance to flexion). Alternately, external sensor may be worn on clothing, such as pants or shirts, adjacent each smart joint. While this example includes joints of the same type, it will be understood that dissimilar smart joints, such as knee 602 and hip 1608 joints can work in tandem in similar fashion.

The “smart” joints may communicate with each other in ways that are similar to how they would communicate with sensors. However, this may entail propagating radio frequency signals from a transmitter inside the body to a receiver that is also inside the body, which may not be efficient. Applicants envision that in addition to the methods described within this specification and others that would be known to those ordinarily skilled in the appropriate art, other means of communication may be deployed. Some examples, without limitation, may include electrically conductive media, such as a wire or wires, that may be subcutaneously inserted to facilitate these communications.

These “smart” joints may also receive configuration commands from external devices and may transmit data to external devices. In one of many non-limiting envisioned embodiments, a person with a “smart” joint may link the “smart” joint to their cell phone 616 or other similar device using known wireless communication and/or telemetry protocols. The cell phone 616 or other device may make use of application software designed and configured to interface with the smart joint. For example, before a patient begins a jog or brisk walk, the patient may “program” a smart joint to a desired level of functionality. Similarly, while jogging or walking, the patient may program or reprogram the smart joint as desired.

As another example, a “smart” joint application may work in tandem with a map feature to geographically locate the person and anticipate their direction and speed and send configuration commands to the “smart” joint. For example, if the map feature indicates that the person is approaching a steep incline, the cell phone may instruct the “smart” joint to set ranges, limits, and biases to appropriate settings for climbing. The ranges, limits, and biases may be kept there until the map feature indicates that the person has moved to different terrain.

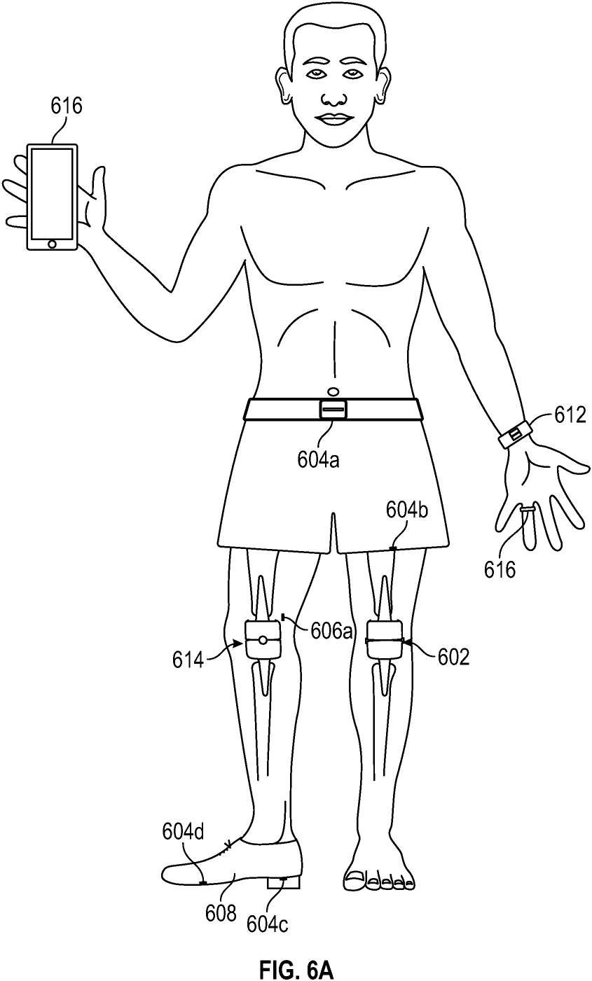

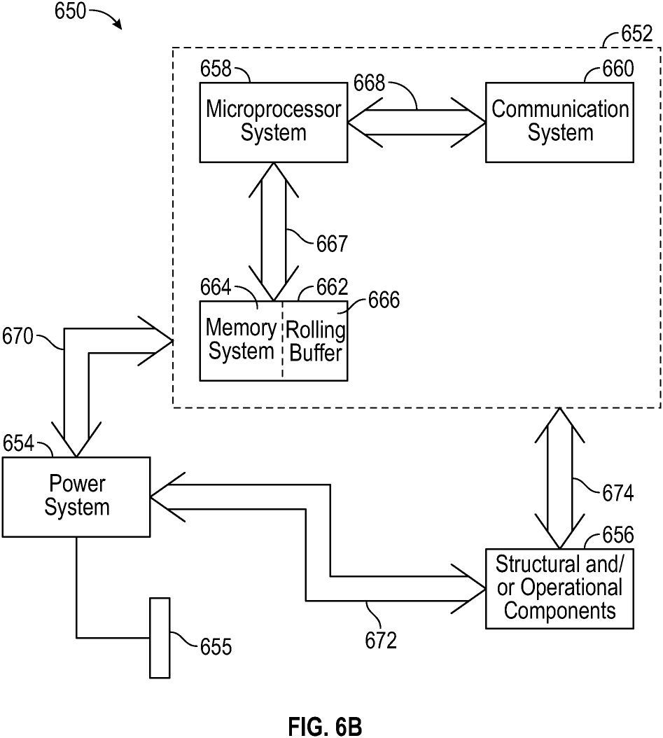

FIG. 6B illustrates an embodiment of a control system 650 comprising a controller 652, a power system 654, the structural and or operational components 656 in the prosthesis that can be adjusted or controlled, such as, without limitation, a prosthetic biasing system and/or a prosthetic resistance mechanism, or both. The controller 652 preferably comprises a microprocessor subsystem 658 configured to execute algorithms (software), firmware and other logic control instructions. The microprocessor subsystem 658 is configured to communicate with a communication subsystem 660, such as over bus 668. Communication subsystem 660 is configured to receive data from one or more internal or external sensors, as described above, and to communicate such data to the microprocessor subsystem 658 over bus 668. The communication system 660 also may be configured to communicate with external smart devices running dedicated application software, as described above. It will be appreciated that the communication protocol implemented and executed by the control system 652 preferably will be a combination of wired and wireless protocols. For example, and not limitation, sensor internal to the prosthesis may be wired to the communication subsystem 660, and sensors external to the prosthesis, such as, but not limited to, belt sensor 604a, may communicate wirelessly.

The controller 652 may comprise a memory subsystem 662 configured to store 664 the prosthetic control and learning algorithms, firmware, and other algorithms, as well as store sensor data and software data, such as generated control instructions. The memory subsystem 662 may preferably comprise a rolling buffer 666 configured to receive the raw sensor data. The algorithms executed by the microprocessor 658 may sample the sensor data in the rolling buffer 666 at periodic intervals. A communication bus 667 transfers data at least between microprocessor 658 and memory subsystem 662.

It is preferred that the controller 652 and power subsystem 654 are integrally associated with the prosthetic body or envelope that is implanted in the human knee space. For example, the controller 652 and power subsystem 654 may be packaged to reside in a portion of the prosthetic stem, or in the prosthetic joint between the stems. Alternately, the control controller 652 and/or power subsystem 654 may be implanted in locations in the body separated from the prosthesis and be operationally connected to the prosthetic knee 656 through wired solutions. For example, and not limitation, the power subsystem 654 may be in an area of the human body that allows better access for power subsystem 654 replacement or recharging. Further still, the power subsystem 654 may comprise a transdermal access port 655 configured to permit recharging of the power subsystem using a smart needle or other such device. Additionally, the transdermal port 655 also may allow transdermal data transfer to the controller 602.

Regardless of where the controller 652 and power subsystems 654 are located, the controller 652 and power subsystem 654 may be configured to communicate data, power, and power and data to one or more of the prosthetic biasing systems and/or prosthetic resistance mechanisms.

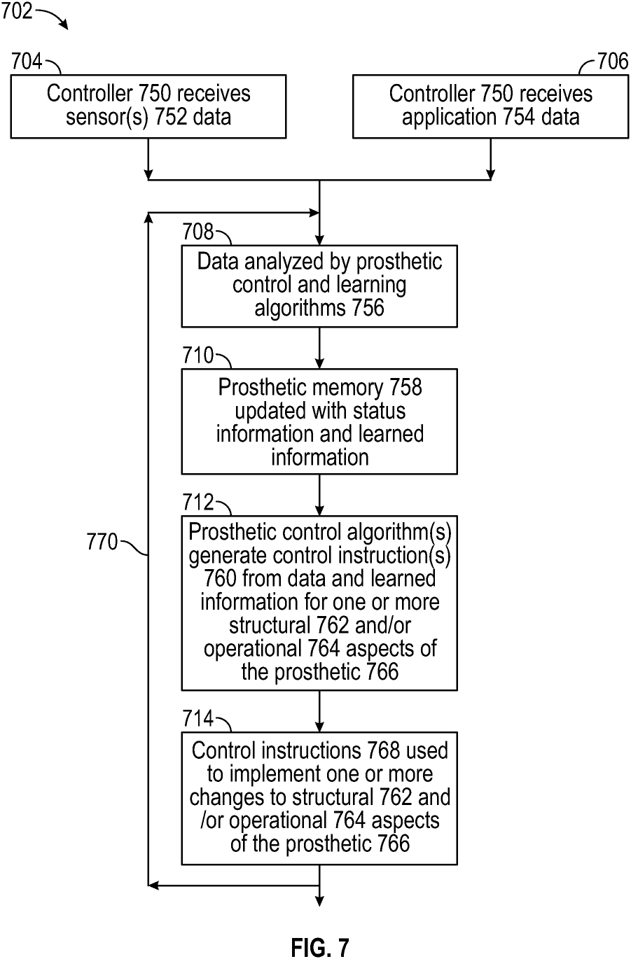

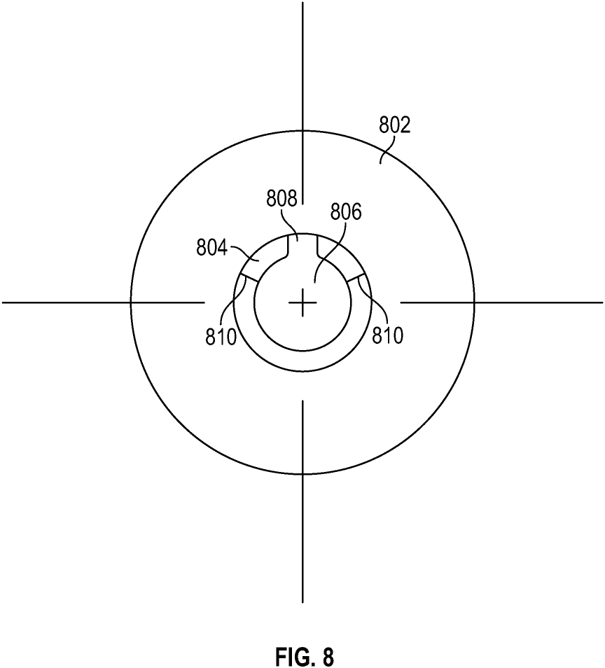

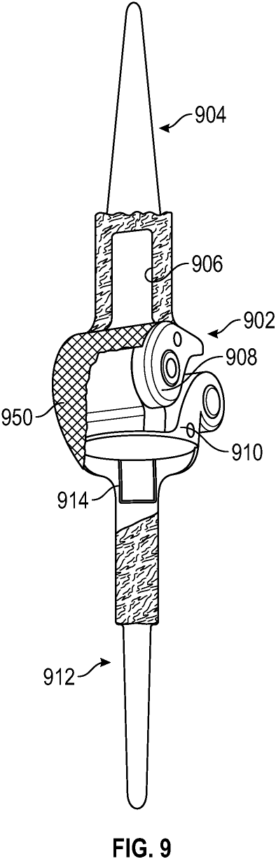

FIG. 7 illustrates one of many algorithms or software useful with embodiments of the present inventions. With non-limiting reference to the embodiment described in FIGS. 6A, 6B and 16, flow chart 702 illustrates the logical progression of a top-level algorithm for a smart prosthesis. The smart joint, such as 602 (knees), 1602 (elbows), 1604 (shoulders), 1606 (ankles) and/or 1608 (hips) comprises a microprocessor or controller 750 configured, such as through software or firmware, to receive information or data from one or more sensors 752. As described previously, these sensor(s) 752 may be external to the prosthetic 766, as such as, but not limited to, a belt sensor 604a, and/or may be an internal sensor associated with the prosthetic, such as, but limited to, an angular position sensor, or an accelerometer. As illustrated at step 704, the controller 750 receives data from one or more of the sensors 752.

As illustrated in step 706, the controller 750 also may be configured, such as through firmware or software, to receive data from an external smart device running a dedicated software application associated with the prosthetic 766. For example, and not limitation, the external smart device may comprise smart phone 616.

As illustrated in step 708, the data, whether sensor data, application data or both, may be analyzed or processed by prosthetic control algorithm 756, which algorithms may or may not comprise machine learning or artificial intelligence learning capabilities. It will be understood by those of skill having benefit of this disclosure that prosthetic control algorithms 756 may be configured and implemented to adjust, vary or control one or more structural or operational characteristics or functions of the prosthetic knee.

In step 710, the memory system 758 of controller 750 may be updated with some or all of the sensor 752 data and/or with status information generated by the prosthetic control algorithms and/or the learning algorithms.

In step 712, the prosthetic control algorithms 756 may generate one or more control instructions 760 from the sensor data 752, application data 754, and/or learned data. The one or more control instructions 760 may be configured to be received by the one or more adjustable or controllable structures or features of the prosthesis, such as, but not limited, the prosthetic extensor mechanism and/or the prosthetic resistance mechanism.

In step 714, the one or more control instructions 768 are used to adjust, vary or control one or more structural 762 or operational 764 characteristics or functions of the prosthetic knee 766.

The algorithm may be configured to loop 770 back to step 708 to analyze new (e.g., new in time) data from the various data sources to determine whether further changes to structural 762 and/or operational 764 characteristics of the prosthesis are needed or desired. For example, and not limitation, if one or more sensor data indicates a period of inactivity over a predetermined period, the algorithms 756 may conclude that the patient is asleep or resting, and the prosthesis and controller 750 may enter a sleep or inactivity mode to conserve power resources.

As disclosed previously and incorporated for this disclosure, the implantable joint preferably comprises a biasing mechanism, which may be a controllable biasing mechanism in a smart embodiment, that biases the joint to the extended position, e.g. 0 degrees flexion, or to some other position, orientation, or alignment. It is preferred that the biasing force (i.e., the force that tends to move the joint to a null position) may be overcome by inertia during movement of the limb. It will be appreciated that too much of a biasing force may simulate a fused joint. Different biasing forces may be provided for various levels of activity. For example, a more active person may require a greater biasing force than a more sedentary person.

It is contemplated that certain embodiments of the joints disclosed herein may have an adjustable or varying biasing force. For example, and not limitation, after implantation, a small tool may be surgically inserted to the joint 602 (knee), 1602 (elbow), 1604 (shoulder), 1606 (ankle) and/or 1608 (hip) to adjust the biasing force, such as by rotating a screw or other component in the joint. As another non-limiting example, a non-surgical example may comprise an external device configured to electronically link to a microprocessor or logic circuit in the joint 602 (knee), 1602 (elbow), 1604 (shoulder), 1606 (ankle) and/or 1608 (hip), such as by magnetic coupling, Bluetooth, or other near field communication protocol, to adjust the bias force or forces, or to adjust operability of the extensor mechanism. For example, a linear motor may be part of the prosthetic elbow and a controller in the joint may adjust the motor to adjust, e.g., the biasing force, or brake force. Alternately, an adjustable rotary orifice may be controlled or adjusted for hydraulic or pneumatic biasing or braking systems in the joint. Alternately, the magnetic direction or strength of implanted magnets may be adjusted or changed to effect a desired bias or resistance to joint movement.

While each of these non-surgical examples has benefits, a practicable solution must prevent unexpected or accidental adjustments. For example, an adjustment made through a magnetic coupling must not be susceptible to accidental changes when the subject is exposed to an otherwise imperceptible magnetic field. Similarly, a Bluetooth interface must not be accessible to unauthorized and/or unauthenticated interference.

Common device authentication and authorization mechanisms known to those ordinarily skilled in the art may be applied to data communications interfaces. As an example, a computerized or “smart” joint may be manufactured with a private encryption key and one or more public keys of the manufacturer. It may be configured to only use encrypted communications such that anyone desiring to communicate with the “smart” joint would then need to use a device that has a key signed by the manufacturer.

Along those lines, a magnetically activated adjustment mechanism may be held in a locked state until an authorized and/or authenticated signal has been received through a data communications link. Alternatively, those ordinarily skilled in the art may make use of multiple magnetic fields to lock and unlock a magnetically activated adjustment mechanism. In one non-limiting example, a fixed field of specific strength oriented in one direction may unlock the mechanism while a movable magnet may associate with the mechanism to make adjustments. Removal of the fixed and oriented field may reengage the lock.

Any of the prostheses disclosed and described herein may comprise a bio sheath, covering, or encapsulation that separates the moving components of the prosthesis from the surrounding soft tissue and vasculature. For example, and not limitation, suitable bio sheaths may include silicon, silicon oxide, silicon nitride, stainless steel mesh or other biocompatible materials. It will be appreciated that the bio sheath, if implemented, should be flexible or deformable enough to accommodate at least the desired range of flexion and rotation, as well as provide the desired amount of separation, encapsulation or protection of the moving components.

Other and further embodiments utilizing one or more aspects of the inventions described above can be devised without departing from the spirit of our inventions. Further, the various methods and embodiments of the methods of manufacture and assembly of the system, as well as location specifications, can be included in combination with each other to produce variations of the disclosed methods and embodiments. Discussion of singular elements can include plural elements and vice-versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

The inventions have been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicants intend to protect fully all such modifications and improvements that come within the scope or range of equivalent of the following claims.

Lastly, the appended claims and what they teach to persons of skill are incorporated into this disclosure for all purposes.

What is claimed is:

1. A prosthesis for replacing a human joint, comprising:
a first component comprising a first prosthetic joint portion and a first intermedullary stem portion, the first stem portion configured to be embedded in a bone associated with the first component;
a second component comprising a second prosthetic joint portion and a second intermedullary stem portion, the second stem portion configured to be embedded in a bone associated with the second component;
a prosthetic joint comprising the first component operatively coupled with the second joint component thereby providing the prosthetic joint with a range of motion of the first component relative to the second component;
a biasing system operatively coupled to the prosthetic joint wherein the first and second components are biased toward a predetermined position when the prothesis is implanted in a human to replace the human joint.

2. The prosthesis of claim 1, wherein the biasing system comprises one or more of a spring system; a magnet system; a hydraulic system; or a pneumatic system.

3. The prosthesis of claim 1, wherein the prosthetic joint is configured to also permit a range of rotation between the first component and the second component.

4. The prosthesis of claim 1, wherein the biasing system comprises a flexion resistance system having at least one friction surface configured provide an increasing amount of resistance to prosthetic joint flexion.

5. The prosthesis of claim 3, further comprising a rotation resistance system having at least one friction surface configured provide an increasing amount of resistance to rotation in either direction from a centered orientation of the prosthetic joint.

6. The prosthesis of claim 1, wherein the prosthetic joint is a monocentric axis flexion joint configured to operate as a simple hinge, or a polycentric axis joint that has multiple pivot points.

7. The prosthesis of claim 1 in which at least a portion of the prosthetic joint is encapsulated in a bio sheath.

8. The prosthesis of claim 1, wherein the stem portion of the first component is removably coupled to the first component for purposes of implantation of the prosthesis.

9. The prosthesis of claim 1, wherein the stem portion of the second component is removably coupled to the second component for purposes of implantation of the prosthesis.

10. The prosthesis of claim 1, wherein the biasing system comprises one or more magnets embedded in the first component and one or more magnets embedded in the second component to attract the first and second components toward a desired orientation or alignment.

11. The prosthesis of claim 1, wherein the biasing system comprises one or more magnets embedded in the first component and one or more magnets embedded in the second component to repel the first and second components form an undesired orientation or alignment.

12. The prosthesis of claim 1, wherein the biasing system comprises a constant friction joint that relies on constant pressure against a rotating surface to resist movement.

13. The prosthesis of claim 1, wherein the biasing system comprises a fluid cylinder configured to bias the joint toward 0° flexion.

14. The prosthesis of claim 13, wherein the fluid is liquid or gas.

15. The prosthesis of claim 14, wherein the biasing system is configured to control the rate of flexion.

16. The prosthesis of claim 1, wherein the biasing system comprises a locking joint configured to simulate a fused joint.

17. The joint of claim 1, wherein the joint comprises a weight-activated control joint.

18. The prosthesis of claim 1, wherein the biasing system comprises one or more magnets embedded in the first component and one or more magnetic materials embedded in the second component to attract the first and second components to a desired orientation or alignment.

19. The prosthesis of claim 1, wherein the biasing system comprises a plurality of magnet sets embedded in the first component and a plurality of magnet sets embedded in the second component to attract and/or repel the first and second components to or form a plurality of desired orientations or alignments.

20. The prosthesis of claim 19, wherein each of the magnet sets are sealed into the first and second components.
